(19) United States
(12) Patent Application Publication
(10) Pub. No.: US 2008/0221564 A1
(43) Pub. Date: Sep. 11, 2008

(54) Method and Device for Identifying and Monitoring a Medical Instrument

(75) Inventors: Jean-Claude Rouiller, La Chaux-de-Fonds (CH); Olivier Breguet, Le Locle (CH); Jacques Suter, Le Locle (CH)

Correspondence Address: DAVIS BUJOLD & Daniels, P.L.L.C., 112 PLEASANT STREET, CONCORD, NH, 03301, US

(73) Assignee: FKG DENTAIRE S.A., La Chaux-de-Fonds (CH)

(21) Appl. No.: 12/088,519

(22) PCT Filed: Sep. 26, 2006
(86) PCT No.: PCT/CH2006/000521

§ 371 Date: Apr. 4, 2008

(30) Foreign Application Priority Data
Sep. 29, 2005 (FR) 05/09973

(51) Int. Cl. A61B 17/32; A61B 18/12; A61B 17/16; A61B 17/00

(52) U.S. Cl. 606/40; 606/167; 606/80

(57) ABSTRACT

A method and device for monitoring and identifying a medical instrument, capable of being incorporated in the instrument itself, and provided with the sufficient storage capacity to store all the data relative to the use of the instrument. The device for monitoring and identifying the medical instrument (10) includes an electronic component (14) integrated in the grip (11) of the instrument. The electronic component is an electronic label comprising an integrated circuit including a storage unit containing variable data based on the use of the instrument (10) and a way for writing and/or modifying and reading the data. The electronic label communicates, via radio frequency, with the software of a motor driving the instrument and via a read/write interface module to allow the software to monitor the motor based on a cyclic fatigue value of the odontological instrument.

### TECHNICAL REALM

The present invention concerns a method for monitoring and identifying a medical instrument, for example a surgical or odontological instrument, comprising at least one grip or handle and one means for intervention on the patient, said intervention means being removable from said grip or said handle in order to perform the operations of cutting tissue, drilling, boring, cauterizing, and the like.

It also concerns a medical instrument, for example a surgical or odontological instrument, comprising at least one grip or handle and one means for intervention on the patient, said intervention means being removable from said grip or said handle in order to perform the operations of cuffing tissue, drilling, boring, cauterizing, and the like, and equipped with a monitoring and identification means for implementing this method.

### PRIOR ART

International Publication WO 01/10329A1 describes a device for monitoring cyclic fatigue in an odontological instrument, one particular embodiment of which comprises an identification code for unmistakably identifying the instrument. Further, this instrument is associated with a storage device recording personalized information consisting of a value representative of cyclic instrument fatigue. This value is updated manually by the user after each use and entered into memory. This modification takes place using a data processing device. The data storage unit and the data processing device are external relative to the instrument.

Accountability standards and the importance of monitoring instruments, especially cyclic instrument fatigue, are growing in response to safety regulations. Existing systems do not offer a simple means of responding to these requirements that is both easy for practitioners to implement and reliable. Additionally, existing systems are cumbersome, preventing the components of such systems from being incorporated in the instruments. Finally, the storage capacity necessary for storing the essential data for each instrument is too large to permit storage in devices small enough to incorporate in most of the actual instruments.

### DESCRIPTION OF THE INVENTION

The present invention proposes a means for overcoming the disadvantages of known systems and furnishes a means for identifying and monitoring a medical instrument, for example a surgical or odontological instrument, that is small enough to incorporate within the instrument itself has sufficient memory to store all the constant data and all the variable data associated with instrument use, and comprises a means for writing and reading this constant data and variable data at the discretion of the user.

This goal is achieved by the method of the invention as defined in the preamble and characterized in that constant data and/or variable data developing over the course of instrument usage is recorded on an integrated storage unit using a means for writing and/or modifying said developing variable data, in that said constant data and said developing variable data is read with data reading devices, and in that said constant data and said developing variable data contained within said data storage unit is detected and transmitted using transmission means.

Advantageously said constant and/or variable data is automatically stored in an integrated memory device and is automatically transmitted to the operating software of a motor driving said medical instrument.

Said constant and/or variable data may be manually recorded in an integrated storage device and automatically transmitted to the operating software of a motor driving said medical instrument.

Preferably said constant and/or variable data is transmitted by radiofrequency to the operating software of a motor driving said medical instrument.

Advantageously said constant and/or variable data is recorded using a read/write interface module.

This goal is also achieved by a medical instrument, for example a surgical or odontological instrument as defined in the preamble and characterized in that said device comprises an electronic element incorporated within the instrument and equipped with at least one data storage unit for storing the constant and/or variable data developing over the course of instrument use, and in that it comprises a means for reading and/or modifying said constant and/or developing variable data, means for writing this constant data and this developing variable data, and a transmission means for detecting and transmitting the constant data and the developing variable data stored in said memory unit.

Advantageously, the electronic element implanted in the instrument is an electronic tag formed of an integrated circuit comprising the storage unit, and the means for reading and writing and/or modifying the data contained in the memory device of the instrument are formed of an interface module.

Preferably the transmission means for detecting and transmitting data is formed of one antenna located in said interface module and one antenna located in the electronic element incorporated within the instrument.

In a first embodiment, said interface module is implanted in a contra-angle connected to the instrument.

In a second embodiment, said interface module is implanted in a data reading/writing device connected to a motor driving the instrument.

Advantageously, said electronic element communicates with the software of a motor driving the medical instrument through a radiofrequency transmission system.

According to variations in embodiments, the data storage unit of the electronic element may be a reprogrammable memory or a read-only memory.

### SUMMARY DESCRIPTION OF THE DRAWINGS

The present invention and its advantages will be more apparent from the following description of preferred embodiments of the invention provided by way of non-limiting example and with reference to the attached drawings, in which:

FIG. 1A is an elevation view of a first embodiment of an odontological instrument equipped with a monitoring and identification device according to the invention;

FIG. 1B is an enlarged view of a portion of the instrument of FIG. 1A;

FIG. 2A is an elevation view of a second embodiment of an odontological instrument equipped with a monitoring and identification device according to the invention;

FIGS. 2B and 2C respectively represent an enlarged elevation and an overhead view of a portion of the instrument of FIG. 2A;

FIG. 3 is a perspective view of a first embodiment of a complete odontological treatment unit;

FIG. 4 is a perspective view of a second embodiment of a complete odontological treatment unit.

### HOW TO ACHIEVE THE INVENTION

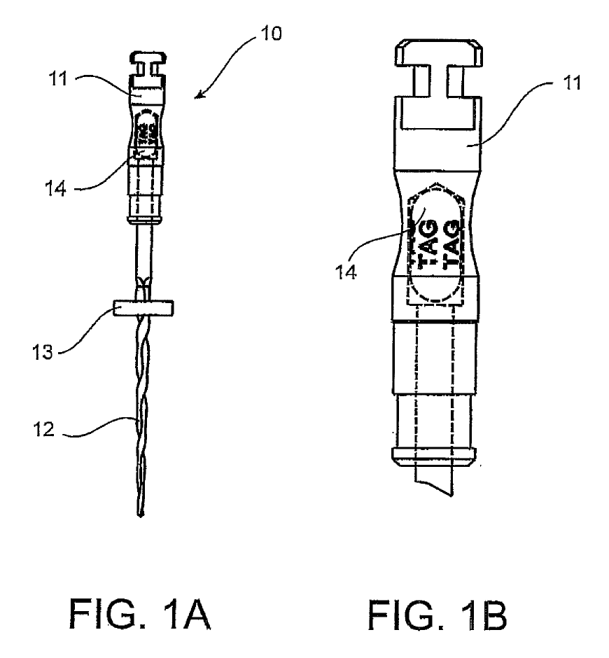

With reference to FIGS. 1A and 1B, the odontological instrument 10 shown comprises a grip or handle 11 and an intervention element 12 constituting the active element, which may consist of a spiral cutting device or a cutting blade, for example. The length of the cutting area the practitioner desires to use to suit the situation is defined by a washer stop 13 movable along the working portion of intervention element 12. Under some circumstances, it may be eliminated at the practitioner's discretion for certain interventions. The spiral cutting device preferably tapers from 2 to 10% and its geometry is defined by the intervention, for example, for dental pulp extraction and shaping the radicular channel.

As FIG. 1B shows more precisely, handle 11 is equipped with a device according to the invention in the form of an electronic element 14 integrated within the body of the handle. This electronic element, currently called a TAG, is an electronic tag composed of an integrated circuit comprising a storage device that can be either a read-only or a reprogrammable memory. This memory device is designed to store the constant data and/or the variable data developing over the course of the use of instrument 10, a means for writing and/or modifying constant data and said developing variable data, a means for reading said constant data and said developing variable data, and a transmission means in the form of an antenna for detecting and transmitting the constant data and the developing variable data contained within said memory device. This electronic TAG device communicates with the software of the instrument's drive motor through a read/write interface module equipped with a transmission means in the form of an antenna. These elements communicate with one another by radiofrequency. The data contained in the TAG allows the software for the drive motor to control the motor as a function of the capital cyclic fatigue of the odontological instrument and, if necessary, to stop the motor and transmit an auditory or visual signal if this capital cyclic fatigue has been surpassed and if continued use of the instrument would present a risk to the patient.

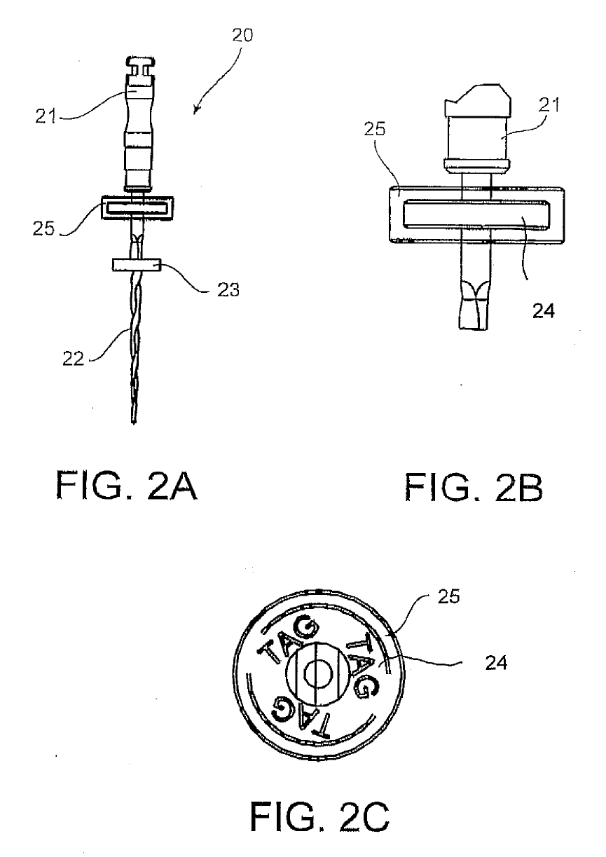

FIGS. 2A through 2C show another embodiment of the odontological instrument equipped with a monitoring and identification device of the invention. This instrument 20 comprises a handle 21, an intervention element 22 constituting the active element and which, like intervention element 12, is in the form of spiral cutting device or a cutting blade. The length of the cutting area the practitioner desires to use for the situation is defined by stop 23, which is movable along the working portion of intervention element 22. This instrument is further equipped with a device 25 for controlling cyclic fatigue in the intervention element 22 as described in International Publication WO 01/10329 A1. In this embodiment, rather than being incorporated within the body of handle 11 like electronic element 14, electronic element 24 is preferably integrated within the cyclic fatigue control device 25 (FIGS. 2B and 2C) of the intervention or in stop 23.

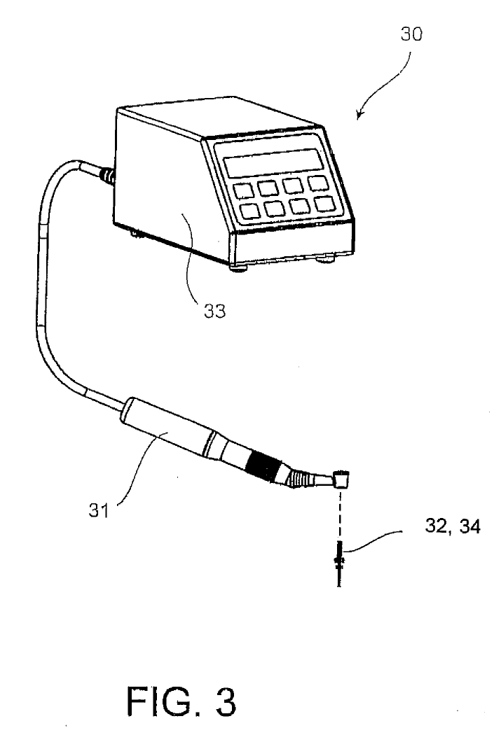

FIG. 3 shows a first embodiment of a complete odontological treatment unit 30 comprising a contra-angle 31, an odontological instrument 32 equipped with an electronic element 34 for introduction inside said contra-angle, and a drive motor 33 for contra-angle 31. According to a preferred embodiment, contra-angle 31 is equipped with an interface module for transmitting directly to the software of drive motor 33 the technical data for the identification code of odontological instrument 32.

In a more complete way, electronic element 34 may be designed to record variable data that develops each time the instrument is used. In a first embodiment, this variable data may be recorded automatically by the electronic element and transmitted to the software for drive motor 34 by means of the read/write module for contra-angle 31. In a second embodiment, this variable data may be recorded automatically by odontological instrument 32 and transmitted through different channels to the software for drive motor 33.

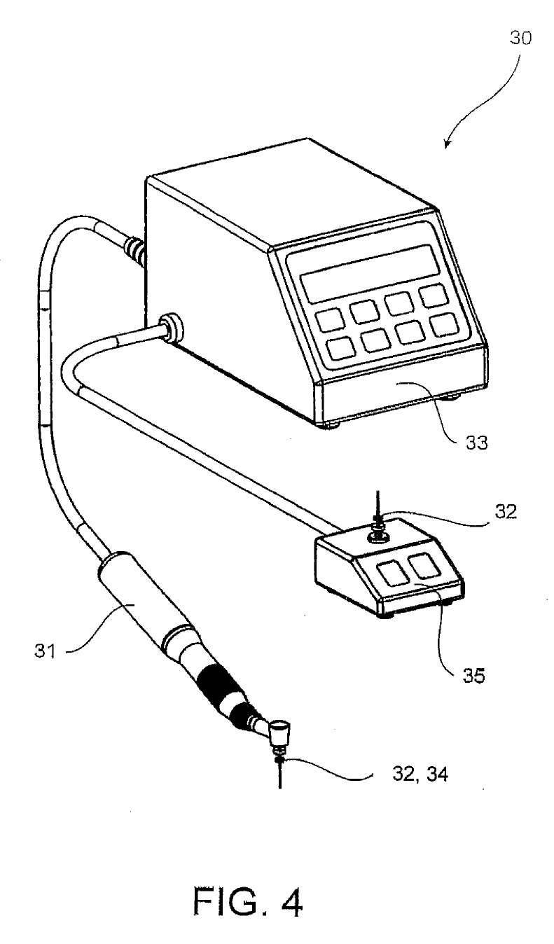

FIG. 4 represents a second embodiment of a complete odontological treatment unit 40 comprising a contra-angle 31, an odontological instrument 32 introduced inside contra-angle 31, and equipped with a device according to the invention comprising an electronic element 34, a drive motor 33 for contra-angle 31, and an external read/write device 35 which receives odontological instrument 32 after use and allows the practitioner to manually record the variable data and simultaneously read the constant data and variable data. In this embodiment the read/write interface module is housed within external apparatus 35.

In a first version, electronic element 14, 24 contains only a single code identifying the instrument 10, 20 which encompasses all the technical data relative to this instrument. For this reason, the drive motor for the instrument is designed to adapt its parameters relative to the data contained in the identification code. These parameters are, for example, rotation speed, working torque, and a parameter called “cyclic fatigue credit” which corresponds to the residual potential reliability of the instrument after each use, said cyclic fatigue credit decreasing in value over the life of the instrument. The data stored in electronic element 14, 24 is constant data which, after transmission to the drive motor, is interpreted by the operating software for the drive motor. Electronic element 14, 24 functions only in read mode. The stored data may be communicated through the usual channels, for example, radiofrequency using an antenna, or by direct connection between instrument 10, 20 and a data reading device connected to the drive motor.

In a second version, electronic element 14, 24 contains on the one hand, a code identifying the instrument 10, 20 which encompasses the technical data relative to the instrument corresponding to the constant data referenced above; and on the other hand, the variable data that develops as a function of wear on the instrument, that is, its cyclic fatigue credit. This cyclic fatigue credit parameter may be expressed as the number of revolutions remaining on an instrument before it must be discarded, or as residual reliable work time. In practice, contra-angle 31 may be equipped directly with a read device for communicating the data to the software for the drive motor. After each use, the new data may be transmitted automatically without any intervention on the part of the practitioner. According to a variation, the contra-angle may be equipped with a manual control that the practitioner activates and which causes the new data to be transmitted to and recorded by the software for the drive motor. According to another variation, the practitioner may manually transmit the data to the specific external data reader 35 whose role is to transmit to and record the new data on the software for the drive motor.

It is possible to envision other embodiments without departing from the scope of the present invention. In particular, the signal devices may be designed to produce an auditory or visual signal when the fatigue credit has been surpassed in light of the fact that accidental breakage of the cutting tool can cause considerable harm to the patient undergoing treatment.

1-15. (canceled)

16. A method for monitoring and identifying a medical instrument having at least one grip or handle (11; 21) and one means for intervention on a patient, the intervention means (12; 22) being removable from the grip or the handle (11; 21) to perform an operation of one of cutting tissue, drilling, boring and cauterizing, and the method comprising the steps of:
recording at least one of constant data and variable data, which develops over a course of instrument use, into an electronic storage unit incorporated within the instrument using a means for at least one of writing and modifying the variable data;
reading the at least one constant data and variable data with a data reading device; and
detecting and transmitting the at least one constant data and variable data recorded into the electronic storage unit using a transmission means.

17. The method according to claim 16, further comprising the step of automatically recording the at least one of constant data and variable data into the electronic storage unit and automatically transmitting the at least one of constant data and variable data to software operating a motor which drives the medical instrument.

18. The method according to claim 16, further comprising the step of manually recording the at least one of constant data and variable data into the electronic storage unit and automatically transmitting the at least one of constant data and variable data to software operating a motor which drives the medical instrument.

19. The method according to claim 17, further comprising the step of transmitting the at least one of constant data and variable data by radio frequency to the software operating the motor which drives the medical instrument.

20. The method according to claim 16, further comprising the step of recording the at least one of constant data and variable data using a read/write interface module.

21. A device for monitoring and identifying a medical instrument (10; 20; 32), having at least one grip or handle (11; 21) and one means for intervention on a patient, the intervention means (12; 22) being removable from the at least one grip or handle to perform at least one operation of cutting tissue, drilling, boring, and cauterizing, and the device comprises:
an electronic element (14; 24; 34), incorporated within the instrument, including at least one data storage unit for storing at least one of constant data and variable data, which develops over a course of instrument use;
a means for at least one of reading and modifying the at least one of constant data and variable data;
a means for writing the at least one of constant data and variable data; and
a transmission means for detecting and transmitting the at least one of constant data and variable data stored in the at least one data storage unit.

22. The method according to claim 21, wherein the electronic element (14; 24; 34), incorporated within the instrument, is an electronic tag with an integrated circuit equipped with the data storage unit.

23. The method according to claim 21, wherein at least one of the means for the at least one of reading and writing and the means for modifying the at least one of constant data and variable data stored in the data storage unit of the instrument is formed of an interface module.

24. The method according to claim 23, wherein the transmission means for detecting and transmitting the at least one of constant data and variable data is formed of an antenna located in the interface module.

25. The method according to claim 21, wherein the transmission means for detecting and transmitting the at least one of constant data and variable data is
